(19) United States
(12) Patent Application Publication
(10) Pub. No.: US 2004/0068233 A1
(43) Pub. Date: Apr. 8, 2004

(54) Venous access device with detachable suture wings

(76) Inventor: Kristian DiMatteo, Watertown, MA (US)

Correspondence Address: TONY ALEXANDER, ESQ, ALVEOLUS, INC. c/o PORTOLIOIP, P.O. BOX52050, MINNEAPOLIS, MN, 55402, US

(21) Appl. No.: 10/264,920

(22) Filed: Oct. 4, 2002

(51) Int. Cl. A61M 5/32

(52) U.S. Cl. 604/177

(57) ABSTRACT

A medical device comprising suture wings that can be detached or attached at various receiving positions about the housing and entry site of the medical device, allows subcutaneous suturing of the device in a manner and at locations as determined by the physician. The suture wings can each comprise one or more suture locations, and can also include a filling material to inhibit tissue ingrowth. If desired, all suture wings can be removed from the medical device. The versatility of the device allows one type of device to be used to accommodate the needs of the different techniques and different methods used by many different physicians.

### FIELD OF THE INVENTION

This invention relates generally to medical devices used to access vasculature.

### BACKGROUND OF THE INVENTION

Long term access to a patient's body is required for many medical treatments including antibiotic therapy, hemodialysis access, chemotherapy regimens, and other treatments that require repeated administration, withdrawal, or exchange of fluids. In some cases, internal access to the patient is required for years.

Subcutaneously implanted access ports are one type of medical device that can be used for this purpose. These medical devices often either include or are attached to a catheter used for the administration and/or withdrawal of fluids from the patient. A pocket is made in the subcutaneous tissue, and the medical device is placed into the pocket. Whether or not the medical device is sutured in place is determined by a number of factors, including the pocket configuration, the type of training received by the physician, and the physician's preference. Once the device is secured, the pocket is closed.

There is a wide range of preferences regarding suture hole presence. Some physicians prefer one, two, three, four, or more suture holes. Some physicians prefer not to have any suture holes present at all, and yet others prefer to have any suture holes that are present filled with a filling material such as silicone to minimize fibrin ingrowth and facilitate the subsequent removal of the device.

What is needed is a medical device and method to allow a physician to conveniently add or remove suture holes to a medical device either prior to or during placement of the medical device within a patient, and that allows suture holes to be located at various preferred locations about the medical device. What is further needed is such a device that optionally comprises a filling material to inhibit fibrin ingrowth into certain of the suture holes.

### SUMMARY OF THE INVENTION

In one aspect, the invention is directed to a medical device for percutaneous access comprising a housing, an entry site, and one or more removable suture wings. The suture wings can be disposed about the housing, at its perimeter, and at other positions, including the entry site. Suture wings removed from the medical device can be reattached, and suture wings can comprise one or more suture locations. The suture wings can be made of polysulfone. DELRIN® (registered mark of E. I. DuPont de Nemours Company Corporation, Wilmington, Del.) can be used for this purpose. The suture wings can also comprise a filling material, to inhibit or promote tissue growth. The entry site can be recloseable, and can comprise a luer or a septum. The entry site can be in fluid communication with a catheter.

Another aspect of the invention is directed to a medical device for percutaneous access to a body comprising an entry site and a housing that is adapted to receive a suture wing in a plurality of receiving positions disposed about the medical device. A received suture wing can be removed and reattached to the medical device. The removed suture wing can be reattached to the position from which it was removed.

Receiving positions for the suture wing can be disposed about the housing, about a perimeter of the housing, and about the entry site. The suture wings can have one or more suture locations, and they can comprise a filling material to promote or inhibit tissue growth. The suture wings can be made of polysulfone. DELRIN® (registered mark of E. I. DuPont de Nemours Company Corporation, Wilmington, Del.) can be used for this purpose. The entry site can be recloseable, and can comprise at least one of a luer and a septum. A catheter can be in fluid communication with the entry site.

### BRIEF DESCRIPTION OF THE DRAWINGS

The foregoing discussion will be understood more readily from the following detailed description of the invention, when taken in conjunction with the accompanying drawings, in which:

FIG. 1 shows a prior art device used to provide access to vasculature;

FIG. 2 illustrates another prior art device with only one entry site;

FIG. 3 shows yet another prior art device with a different type of an entry site and a closing feature;

FIG. 4 is an illustration of one exemplary embodiment of the invention;

FIG. 5 illustrates another exemplary embodiment of the invention that includes a suture wing disposed near the entry site;

FIG. 5A shows a cross-sectional view of one exemplary embodiment of the suture wing of FIG. 5;

FIG. 5B shows a cross-section of a portion of the tube of FIG. 5;

FIG. 6 illustrates a cross-sectional view of one exemplary embodiment of a suture wing of the invention;

FIG. 7 is a cross-sectional view of the housing of one exemplary embodiment of the invention;

FIGS. 7A and 7B show the cross-section of different exemplary embodiments of suture wings;

FIG. 8 is a perspective view of the underside of the housing of the medical device that can be used with the suture wings of FIGS. 7A and 7B;

FIGS. 8A and 8B are cross-sectional views of different embodiments of the housing of the medical device;

FIG. 8C is a bottom view of an exemplary embodiment of the medical device that comprises discrete suture wing attachment locations;

FIGS. 9A and 9B show different exemplary embodiments of the suture wings that can be attached using a friction fit with a housing, such as that illustrated in FIG. 8A;

FIGS. 10A and 10B are plan views of exemplary suture wings comprising more than one suture location; and

FIG. 11 shows a suture location comprising filling material.

### DETAILED DESCRIPTION

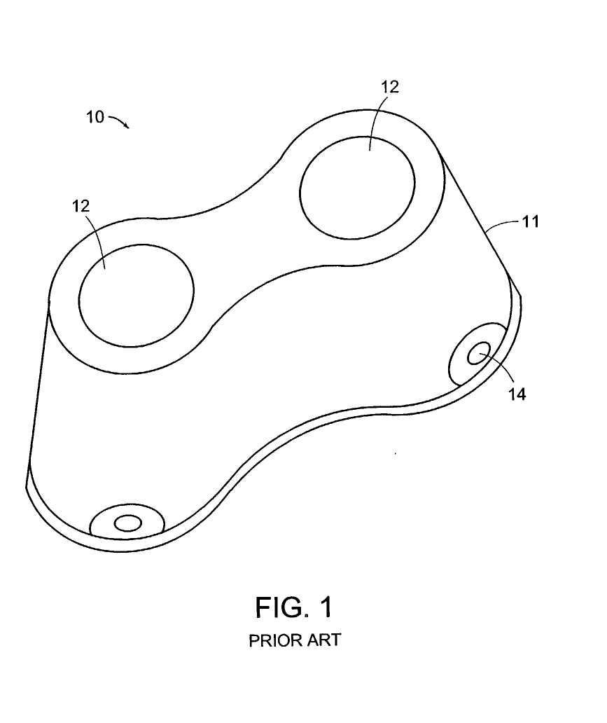

Referring to the drawings, wherein like reference numbers refer to like parts, FIG. 1 shows a prior art device used to provide access to vasculature. The medical device 10 includes a housing 11 and an entry site 12 for the administration, withdrawal, or exchange of fluids. The entry site can be a septum, and the needle of a syringe, for example, can be used to administer or withdraw fluids through the septum. The medical device can be surgically implanted beneath the skin of a patient.

The medical device of FIG. 1 can also include suture locations 14 disposed about the base of the housing and disposed in fixed, permanent locations. The suture locations can be molded into the body 11 as shown in FIG. 1. A physician can tie a suture through the suture hole, thus securing the medical device 10, for example, to adjacent tissue. FIG. 1 illustrates a medical device with two entry sites.

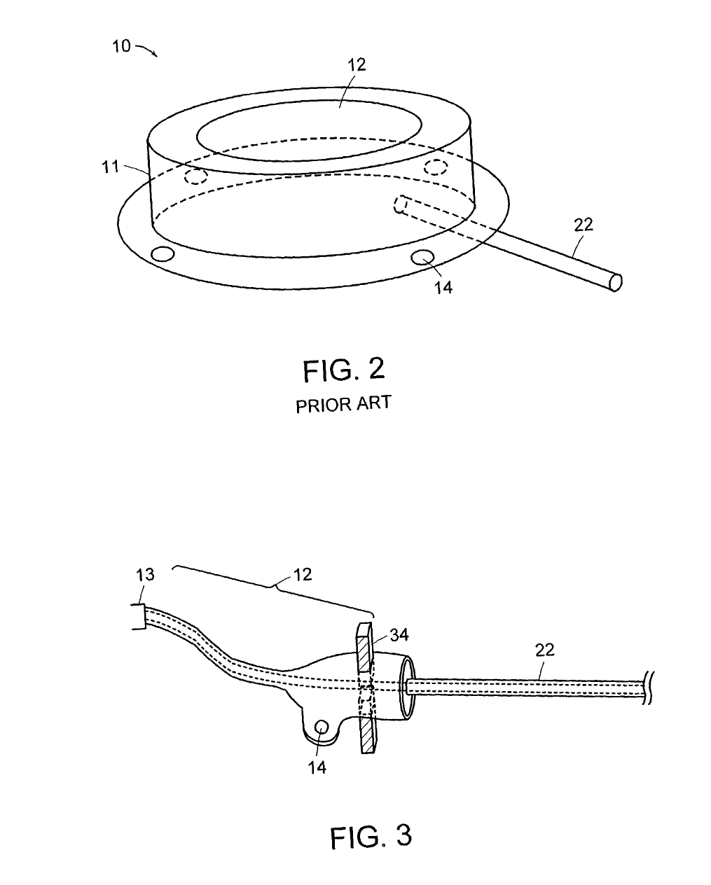

FIG. 2 illustrates another prior art device, with only one entry site 12. A catheter 22 is attached to the medical device 10. Suture locations 14 are disposed about the base of the housing 11, again in permanent, fixed locations.

FIG. 3 shows yet another prior art device with a different type of an entry site 12 and a closing feature. The suture location 14 is attached to a side of the housing 11. The entry site 12 can include a luer connection 12A disposed at an end of the entry site. This medical device 10 comprises a closing mechanism 34 that can be used to restrict or shut off fluid flow through the device 10, as required. Use of the closing mechanism 34 results in an entry site 12 that is recloseable.

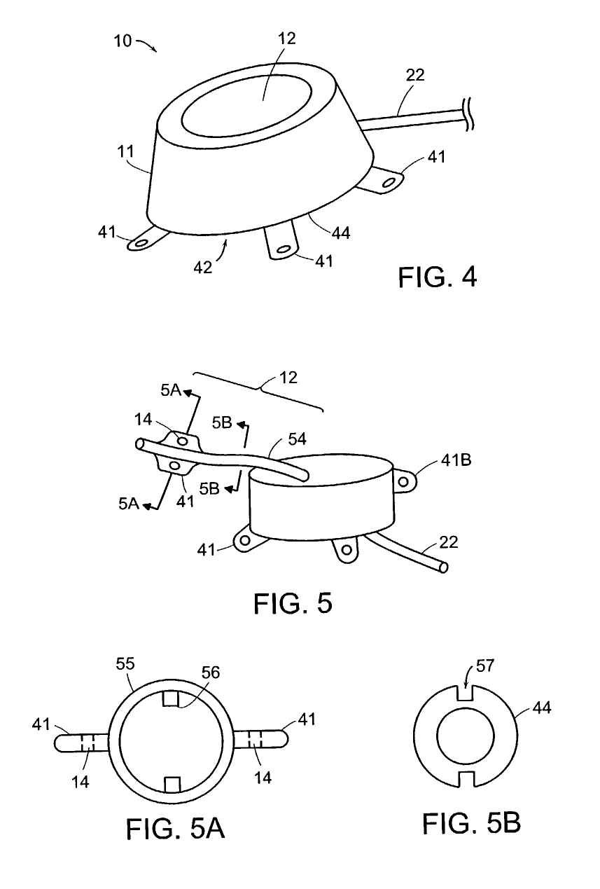

FIG. 4 is an illustration of one exemplary embodiment of the invention. The medical device 10 comprises housing 11 and an entry site 12. Several suture wings 41 are disposed about the base 42 of the device 10. The suture wings can be disposed at the perimeter 44 of the base 42. These suture wings 41 are adapted to be removable from the medical device 10. They can be removed by different methods and mechanisms, described in more detail below.

The medical device of FIG. 4 comprises a plurality of suture wings 41, however, a single suture wing is contemplated in certain other embodiments. In this particular embodiment of the invention the suture wings 41 are disposed externally to the base 42. They can, however, be integrally molded with the housing, in which case they possess a breakaway feature that allows one or more of them to be removed by applying a downward force, and snapping them off of the housing 11. Generally, if the suture wings 41 are integrally formed with the base, they are not mechanically reattachable. In other embodiments of the invention, described in more detail below, suture wings 41 may be repeatedly attached to and removed from the housing 11.

In this exemplary embodiment of the invention both the housing and the suture wings can be made of the same material. For example, they can be made of polysulfone. DELRIN® (registered mark of E. I. DuPont de Nemours Company Corporation, Wilmington, Del.) can be used for this purpose. Not all embodiments of the invention require the housing and the suture wing to made of the same material, although they generally are made of the same material when they are integrally formed together.

FIG. 5 illustrates another exemplary embodiment of the invention that includes a suture wing disposed adjacent the entry site 12. The entry site comprises a tube 54 that can be formed of a flexible material, although the configuration of the entry site can be varied, as will be appreciated by the artisan of ordinary skill in the art. The entry site can alternatively comprise a luer connection 12A. FIG. 5 illustrates two suture wings 41 disposed on the entry site, although any number can be accommodated. The suture locations 14 of these suture wings can be used by a physician to secure the location of the entry site within the body of a patient. This is particularly useful if the tube 54 of the entry site 12 is overly flexible. As discussed above, suture wings can be disposed about the housing 11 of the medical device 10, including on the side of the housing as illustrated by suture wing 41B. The housing can further comprise a catheter 22.

FIG. 5A shows a cross-sectional view of one embodiment of the suture wing of FIG. 5. This view shows the cross-section as illustrated by section line 5A of FIG. 5. This exemplary embodiment of the invention includes a collar 55 comprising one or more slots 56 adapted to mate with the tube 54. In this embodiment of the invention it is desirable that once attached, the suture wing be restrained from rotating about the circumference, to provide improved placement stabilization of the entry site 12. The collar can comprise one or more suture wings 41. Mechanical means other than or in addition to the slot 56 may be used to attach the collar 55 to the tube 54. For example, snaps or biocompatible adhesive may be used. Further, the collar 55 can be configured to open and close, and various methods and devices to secure the collar in a closed position can be employed. Although this figure shows the collar attached to the entry site, the same techniques can be used to attach it to other portions of the housing 11.

FIG. 5B shows a cross-section of a portion of the tube of FIG. 5, as illustrated by sectional line 5B. This cross-section illustrates a recess 57 into which the slot 56 of collar 55 can be inserted. The recess 57 can be in the form of a groove that runs along the length of tube 54. However, it is not necessary for the recess 57 to be a groove, and alternative configurations will be appreciated by the artisan of ordinary skill.

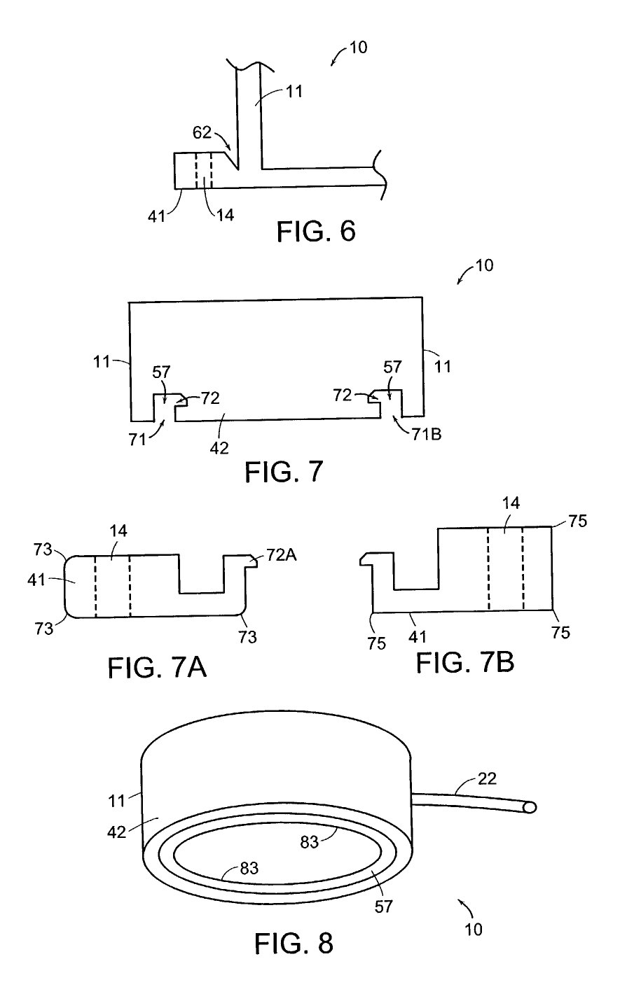

FIG. 6 illustrates a cross-sectional view of one exemplary embodiment of a suture wing of the invention. This suture wing 41 comprises one suture location 14 and is integrally molded with the medical device 10. As shown in this figure, it is integrally molded with the housing 11. A chamfered edge 62 of the suture wing 41 can be used to provide for convenient and easy removal of the suture wing from the medical device 10, if removal is desired. The chamfered edge 62 also provides for a clean break when the suture wing 41 is removed, minimizing or eliminating rough surfaces or appendages that might cause injury or irritation to a patient. Preferably, the chamfered edge 62 is created in such a manner that the suture wing can be removed without the use of tools. A smooth and pleasing exterior surface is presented after removal of the suture wing 41, due in part to the presence of the chamfered edge 62. As illustrated, the suture wing 41 of FIG. 6 comprises a single suture location 14, although multiple suture locations can be accommodated.

FIG. 7 is a cross-sectional view of the housing of one exemplary embodiment of the invention, illustrating receiving positions 71 in the medical device. In this embodiment, receiving positions comprise recesses 57 and lips 72. Although the illustration shows recesses 57 located in the base 42 of the housing 11, the suture wing attachment techniques described here and elsewhere can be employed at virtually any location on the medical device 10. The medical device 10 of FIG. 7 is adapted to receive suture wings at a plurality of receiving positions 71. In the embodiment shown, recesses 57 comprise a lip 72 adapted for receiving one or more suture wings.

FIGS. 7A and 7B show the cross-section of other exemplary embodiments of suture wings. The suture wings 41 of FIGS. 7A and 7B each comprise a lip 72A adapted to mate with the lip 72 of the recess 57 of FIG. 7. The suture wing 41 and the medical device 10 may be joined together using this snapping feature. When the lip 72A of the suture wing 41 is inserted into the recess 57, it mates with the lip 72 of the recess 57, thereby attaching the two pieces together. This figure illustrates but one of the many mechanical snapping methods that are available and known to those of one of ordinary skill in the art for attaching one piece, such as a suture wing, to another piece, such as the housing 11 of medical device 10. Various mechanical fastening methods can be used, but a snapping/unsnapping method is generally preferred.

After the suture wing 41 is attached to the medical device 10 by any of these methods, it can subsequently be removed from the medical device by applying physical force to separate the suture wing 41 from the medical device 10, to remove it from the recess 57. In this manner, the suture wing may be attached, detached, and reattached to the medical device. When the suture wing is reattached it can be located at a new receiving position 71B, or returned to the original receiving position 71, as the physician prefers.

Biocompatible adhesive materials can also be used to attach the suture wings 41 to the medical device 10, either in addition to the mechanical methods described above, or in place of them. However, these adhesives should not be used if the suture wings 41 are to be removed and subsequently reattached to the medical device. Alternatively, biocompatible adhesives with low or moderate tacking strength can be used to permit repositioning of the suture wings 41.

Further, for any embodiment of the invention attaching of the suture wings can include the use of snaps, fasteners, a friction fit, coupling, joining, connecting, adhesives, interlocking mechanisms, various securing means, joining, affixing, and the like. Removal of the suture wings from the medical device includes various techniques by which they can be unfastened, unsnapped, detached, and the like.

The suture wings 41 illustrated in FIGS. 7A and 7B exhibit different surface contours. The suture wing of FIG. 7A presents rounded surfaces 73 at its exposed points, whereas the suture wing of FIG. 7B has square edges 75. Generally, rounded edges are preferred to minimize irritation and patient discomfort, although both embodiments are within the scope of the invention. Selection of the type of suture wing to be used is determined by the preference of the physician. The medical device 10 can comprise different suture wing styles, at the discretion of the physician.

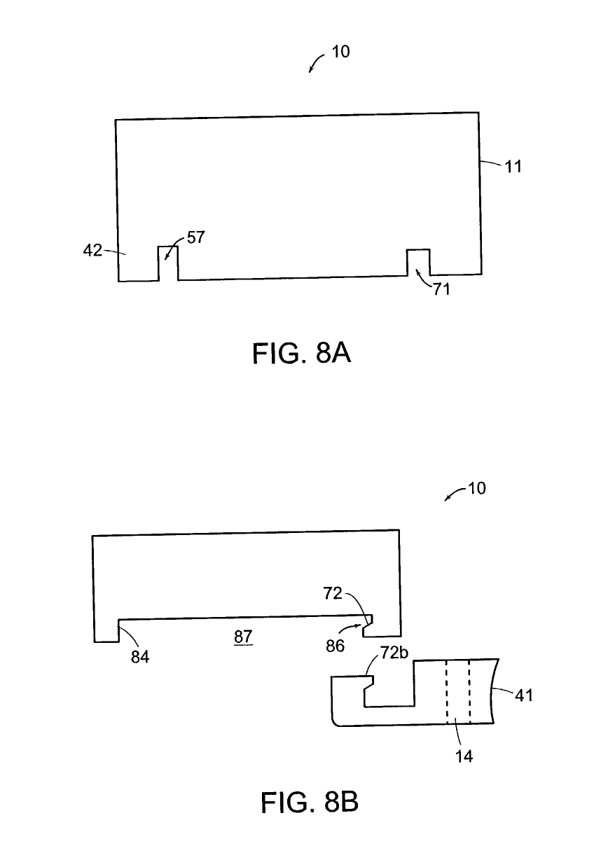

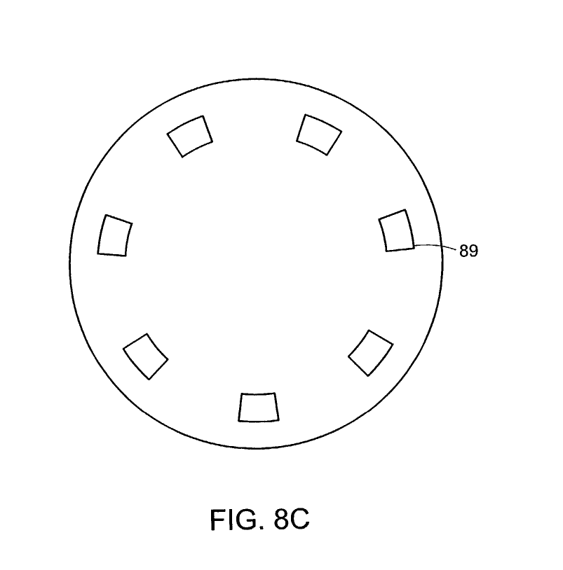

FIG. 8 is a perspective view of the underside of housing 11 of medical device 10 that can be used, for example, with the suture wings of FIGS. 7A and 7B. In this embodiment of the invention, recess 57 forms a contiguous groove 83 about the base 42 of the medical device. The contiguous groove 83 defines a plurality of receiving positions 71 to which suture wings 41 may be attached. In some embodiments of the invention, the suture wings may then be detached, and reattached to the same or different receiving locations. Although the contiguous groove 83 is illustrated as being on the base 42 of housing 11, it can be located on other portions of the medical device 10.

Further, the contiguous groove does not need have a circular shape. For example, the contiguous groove can be in the shape of a box, a cross, or various other geometric shapes. Other shapes of the contiguous groove are within the scope of the invention.

FIGS. 8A and 8B are cross-sectional views of different embodiments of the housing 11 of medical device 10. Although the recess 57 in FIG. 8A is illustrated as being located on the base 42 of housing 11, it can be located on other portions of the medical device 10. In the embodiment shown in FIG. 8A, the recess 57 is disposed at an internal location in the medical device, but without lip 72. Lip 72 is not a required feature of the invention. As described above, recess 57 of FIG. 8A can comprise a contiguous groove 83 disposed about various locations on the medical device 10, and this contiguous groove can have many different shapes and configurations.

FIG. 8B illustrates a cross-sectional view of other exemplary embodiments of the medical device 10. A receiving position without a mechanical interlock 84 is illustrated, as is another embodiment with a mechanical interlock 86, including lip 72. Both receiving positions are adapted to receive one or more suture wings 41. The receiving position with the mechanical interlock 86, or without the mechanical interlock 84, can each be part of a contiguous groove 83 disposed about the medical device, as described above. As illustrated in FIG. 8B, both types of receiving positions are externally located on the medical device, as contrasted with the internal recess shown in FIG. 8A. Combinations of all of these various features can also be used.

As illustrated in FIG. 8B, the central area 87 can be a void, reducing the amount of material required to form the medical device. Suture wings 41, such as those illustrated in FIGS. 7A and 7B, can both be used with receiving positions such as 84 and 86. Preferably, the suture wing style used will conform to the receiving location style. For example, the suture wing lip 72B is in a preferred orientation for use with receiving location 86, such that this receiving position and suture wing will most efficiently mate with each other. In addition to mechanical interlocking methods, such as the one illustrated at receiving position 86, other alternative attachment methods are within the scope of the invention. For example, a friction fit can be used to attach a suture wing 41 to receiving position 84 or receiving position 86. Biologically compatible adhesives can also be used, and are within the scope of the invention. Optionally, biocompatible adhesives with low or moderate tacking strength can be used to permit repositioning of the suture wings 41.

FIG. 8C is a bottom view of an embodiment of the medical device that comprises discrete suture wing attachment locations 89. These discrete suture wing attachment locations can be disposed at various locations about the medical device 10, such as at the base 42, the perimeter 44, or the entry site 12. The discrete suture wing attachment locations can be adapted to receive suture wings such as those illustrated in FIGS. 5A, 7A, 7B, 8B, 9A, and 9B.

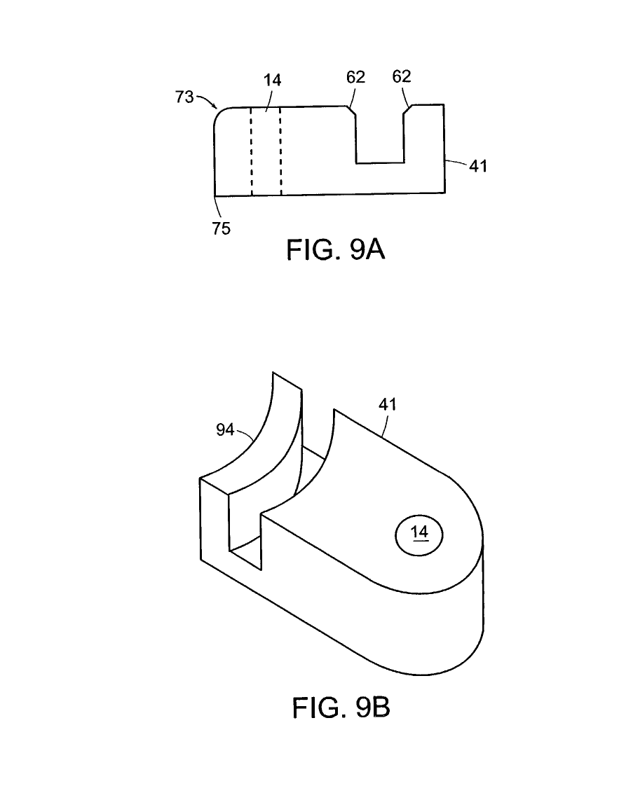

FIGS. 9A and 9B show different embodiments of the suture wings 41 that can be attached to the medical device 10 using a friction fit. For example, in a preferred embodiment the suture wing of FIG. 9A is used with a medical device as illustrated in FIG. 8A. As shown in FIG. 9A, the suture wing can comprise rounded edges 73, square edges 75, and chamfered edges 62. Different combinations of these edges can be used. Use of one or two chamfered edges 62 aids in the alignment of the suture wing 41 with the selected receiving position (e.g., 71 or 89). The suture wing 41 of FIG. 9B has a curvature 94 that is adapted to conform, for example, to the curvature of the contiguous groove 83 of FIG. 8. This embodiment of a suture wing 41 does not have a mechanical interlock, and is adapted to efficiently attach to a receiving position such as receiving position 84, illustrated in FIG. 8B. This attachment is preferably accomplished with a friction fit, and optionally, also using a biocompatible adhesive material. If no adhesive is used, the suture wing may conveniently be detached and reattached, if desired.

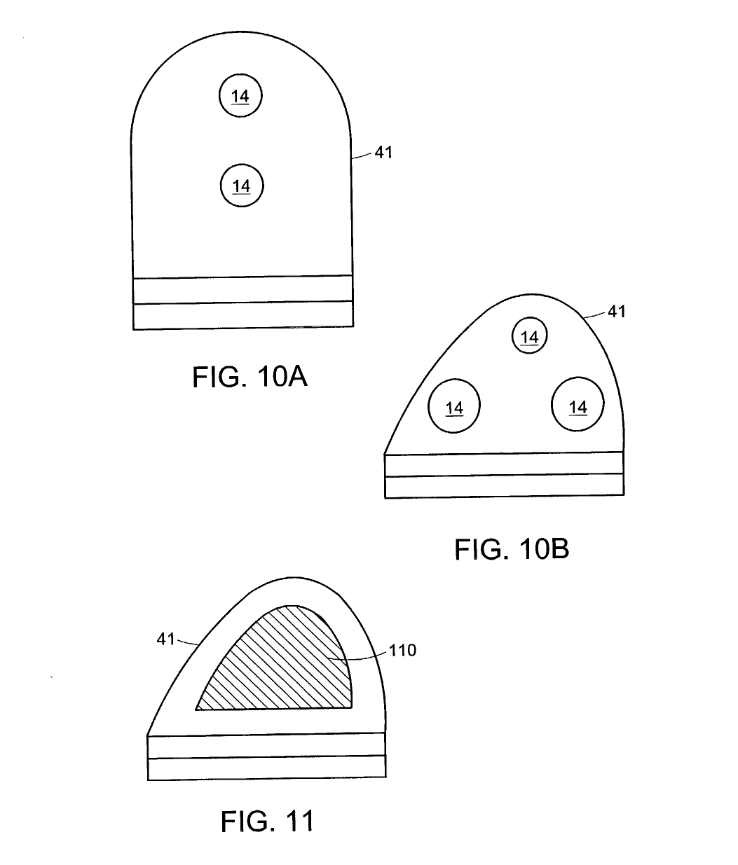

FIGS. 10A and 10B are plan views of suture wings comprising more than one suture location 14. Suture wings can comprise different numbers of suture locations 14, and they can be in different configurations and orientations. Although these suture locations are illustrated having a circular cross-section, other geometries can be used and are within the scope of the invention.

FIG. 11 shows a suture location comprising filling material 110. In all embodiments of the invention, filling materials can be used to inhibit the ingrowth of tissue materials. Removal of the medical device is thereby facilitated, in that tissue growth has occurred only at desired locations about the medical device. For example, suture locations 14 that are not removed from the medical device 10 can be filled with silicone, preventing tissue ingrowth. Thus, fibrin will not grow into these suture locations, and they can later be more easily removed. Of course, suturing can still be performed even using suture locations 14 that are filled or pre-filled with silicon. Other materials can be used to fill the suture locations 14, such as CORETHANE® (registered mark of Corvita Corporation, Miami, Fla.), polyurethane, and bionate polycarbonate urethanes. Materials with a Durometer reading of 50-55 are preferred for this purpose. Such materials are relatively soft, and can be conveniently punctured with a needle. An appropriate material thickness should be used, as is apparent to one of ordinary skill in the art.

Conversely, materials that promote tissue growth can also be used on portions of the medical device 10. When such materials are used, they are generally secured to a surface of the medical device 10.

Various methods can be used to secure the medical device 10 within a patient. In one embodiment, the medical device 10 is implanted into a patient as follows. First, the surgeon determines the location and number of suture wings desired on the medical device 10. If suture wings are present on the medical device, the undesired suture wings are removed. If insufficient suture wings are present, more are added. The suture wings added can have one or more suture locations, depending upon the preference of the physician.

A linear incision is made in the patient. The catheter 22 and the housing 11 are inserted through the incision, into the patient, and near an area in which the catheter 22 is to be placed. After these are positioned, subcutaneous sutures are sewn through the suture locations 14, securing the suture wings 41 and the medical device 10 to adjacent tissue. These sutures are used to anchor the subdermal layers to the medical device 10. In other embodiments, subcutaneous hooks may be used to anchor the subdermal layers to the medical device 10.

In some embodiments, the suture wings 41 or the medical device 10 can be coated with materials that promote tissue growth to provide better sealing of the incision, such as collagen or other tissue growth catalysts. Materials that promote ingrowth of cells, such as a permeable fabric, a textured polymer, or appropriate mesh materials can also be bonded to or embedded into the surface of medical device 10 or suture wings 41. The added ingrowth materials cause the skin surrounding the medical device 10 to bond securely with the medical device 10. Alternatively, growth inhibition materials can be placed on or around the medical device. For example, suture locations 14 that will not be used can be filled with silicone to prevent tissue ingrowth. Thus, fibrin will not grow into these suture locations, and they can later be more easily removed.

After proper placement of the catheter 22, and securing of the medical device 10, the incision area can then be closed and bandaged.

While the invention has been particularly shown and described with reference to specific preferred embodiments, it should be understood by those skilled in the art that various changes in form and detail may be made therein without departing from the spirit and scope of the invention as defined by the appended claims.

What is claimed is:

1. A medical device for percutaneous access to a body comprising:
a housing;
an entry site for the administration, withdrawal, or exchange of fluids; and
one or more removable suture wings.

2. The medical device of claim 1 wherein one or more of the suture wings are disposed about the housing.

3. The medical device of claim 2 wherein one or more of the suture wings are disposed about a perimeter of the housing.

4. The medical device of claim 1 wherein at least one suture wing is disposed about the entry site.

5. The medical device of claim 1 wherein at least one of the one or more suture wings is adapted to be removed and reattached to the medical device.

6. The medical device of claim 1 wherein at least one of the one or more suture wings comprises one or more suture locations.

7. The medical device of claim 1 wherein at least one of the one or more suture wings comprises a filling material adapted to inhibit tissue ingrowth.

8. The medical device of claim 1 further comprising tissue ingrowth material.

9. The medical device of claim 1 wherein the suture wings comprise polysulfone.

10. The medical device of claim 1 wherein the entry site is recloseable.

11. The medical device of claim 1 wherein the entry site comprises at least one of a luer and a septum.

12. The medical device of claim 1 further comprising a catheter in fluid communication with the entry site.

13. A medical device for percutaneous access to a body comprising:
an entry site for the administration, withdrawal, or exchange of fluids; and
a housing, wherein the housing is adapted to receive at least one suture wing in or at a plurality of receiving positions disposed about the medical device.

14. The medical device of claim 13 wherein a received suture wing can be removed from a first position.

15. The medical device of claim 14 wherein the received suture wing is adapted to be reattached.

16. The medical device of claim 15 wherein the received suture wing is adapted to be reattached to the first position.

17. The medical device of claim 13 wherein one or more of the plurality of receiving positions is disposed about the housing.

18. The medical device of claim 17 wherein one or more of the plurality of receiving positions is disposed about a perimeter of the housing.

19. The medical device of claim 13 wherein at least one of the plurality of receiving positions is disposed about the entry site.

20. The medical device of claim 13 wherein at least one suture wing is adapted to be received by and then removed from one or more of the plurality of receiving positions.

21. The medical device of claim 13 wherein at least one suture wing comprises one or more suture locations.

22. The medical device of claim 13 wherein at least one suture wing comprises a filling material adapted to inhibit tissue ingrowth.

23. The medical device of claim 13 further comprising tissue ingrowth material.

24. The medical device of claim 1 wherein at least one suture wing comprises polysulfone.

25. The medical device of claim 13 wherein the entry site is recloseable.

26. The medical device of claim 13 wherein the entry site comprises at least one of a luer and a septum.

27. The medical device of claim 13 further comprising a catheter in fluid communication with the entry site.
